(19) United States
(12) Patent Application Publication
(10) Pub. No.: US 2024/0424117 A1
(43) Pub. Date: Dec. 26, 2024

(54) Paclitaxel Prodrug, Preparation Method and Application Thereof

(71) Applicant: WEST CHINA HOSPITAL, SICHUAN UNIVERSITY, Chengdu (CN)

(72) Inventors: Ruichao LIANG, Chengdu (CN); Lu SUN, Chengdu (CN)

(21) Appl. No.: 18/338,477

(22) Filed: Jun. 21, 2023

(51) Int. Cl. A61K 47/64; A61K 9/107; A61K 47/54; A61K 47/60

(52) U.S. Cl. CPC A61K 47/64; A61K 9/1075; A61K 47/542; A61K 47/60

(57) ABSTRACT

A paclitaxel prodrug, includes a disulfide bond, a PEG molecular chain, and a RGD peptide chain in the structure. The paclitaxel prodrug can self-assemble into micelles in an aqueous environment, with good in vitro stability. The introduced disulfide bond is conducive to the specific bond-breaking of the prodrug at the tumor sites, that can cause the disintegration of the prodrug micelles, damage the micelle morphology, and thus exhibit good sustained-release effects.

### TECHNICAL FIELD

The present invention belongs to the field of medicinal chemistry, and in particular, relates to a paclitaxel prodrug, preparation method therefor and use thereof.

### BACKGROUND TECHNOLOGY

Cancer is one of the main causes of death for people around the world. In the past few decades, chemotherapy has been widely used as a potential and recognized treatment method for local and metastatic cancers, and the clinical application of anti-tumor drugs has greatly extended the lives of patients with cancers.

Paclitaxel (PTX) is a natural product with anti-cancer activity. Currently, it has become the third generation of anti-tumor drug following doxorubicin and cisplatin, and demonstrated good therapeutic effects on ovarian cancer, breast cancer, prostate cancer, lung cancer, and so on. However, there still are many challenges for the application of paclitaxel as an anticancer drug. Firstly, it has certain toxicity to normal and healthy proliferative cells, which is also a common challenge for current chemotherapy drugs. Secondly, paclitaxel has a poor water solubility. At present, polyoxyethylene castor oil is used as the solvent for the paclitaxel preparations in clinical use, which has certain defects such as high toxicity and side effects.

Therefore, improving the drug system, solving the problem of low solubility of paclitaxel, enhancing its targeting ability towards tumor tissues, and limiting its side effects on healthy tissues are the key to its successful use, as an anticancer drug, for the treatment of cancer.

Prodrugs refer to chemical derivatives of active pharmaceutical ingredients, which are generally inactive and can be converted into pharmaceutical ingredients in the body to exert therapeutic effects. Compared with free drug loaded liposomes, micelles, nanoparticles and other types of dosage forms, prodrugs are prepared by chemical bonding, with better quality control and small batch changes. PTX prodrugs are usually constructed on the 2′-OH or 7-OH positions of PTX. Compared to the 7-OH position, the 2′-OH position of PTX is more suitable for chemical reactions to reserve the cytotoxicity, and the prodrugs can be synthesized without protecting the 7-OH position. At present, it has been reported in the literature that the succinyl group is linked to the 2′-OH position of PTX, and then conjugated with PEG (MW 5000) to improve the solubility of PTX, showing in vitro cytotoxicity equivalent to PTX against melanoma cells (Li C, Yu D F, Inoue T, et al. Synthesis and evaluation of water-soluble polyethylene glycol-paclitaxel conjugate as a paclitaxel prodrug [J]. Anti-Cancer Drugs, 1996, 7 (6): 642-8).

In order to improve the solubility of paclitaxel as well as enhance its targeting effect on tumor tissues, further exploration of paclitaxel prodrugs is of great significance.

### SUMMARY OF THE INVENTION

The object of the present invention is to provide a paclitaxel prodrug that can spontaneously form a micelle structure to enhance the solubility of paclitaxel and has tumor-targeting properties.

The present invention provides a paclitaxel prodrug, which comprises a disulfide bond, a polyethylene glycol molecular chain, and an RGD peptide chain.

Further, the structure of the aforementioned paclitaxel prodrug includes a cyclic peptide moiety with the sequence RGDCF.

More further, the structure of the aforementioned paclitaxel prodrug is as follows:

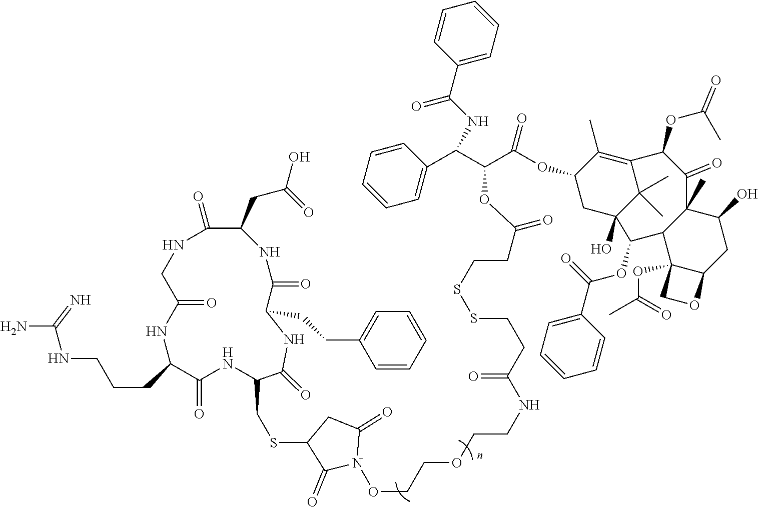

wherein n is any value between 40 and 50.

More further, the number-average molecular weight of the polyethylene glycol (PEG) molecular chains is 2000.

The present invention also provides a preparation method for the paclitaxel prodrug, comprising the following steps:
(1) Paclitaxel reacts with dithiodipropionic acid anhydride in an organic solvent in the presence of an activator and a stabilizer, to prepare PTX-DTPA;
(2) PTX-DTPA reacts with Maleimide-PEG-Amine in an organic solvent in the presence of an activator, a stabilizer and a condensation agent, to prepare PTX-DTPA-PEG;
(3) PTX-DTPA-PEG and cyclic peptide RGDCF react in aqueous solvents, to prepare the paclitaxel prodrug; The reaction formula is as follows:

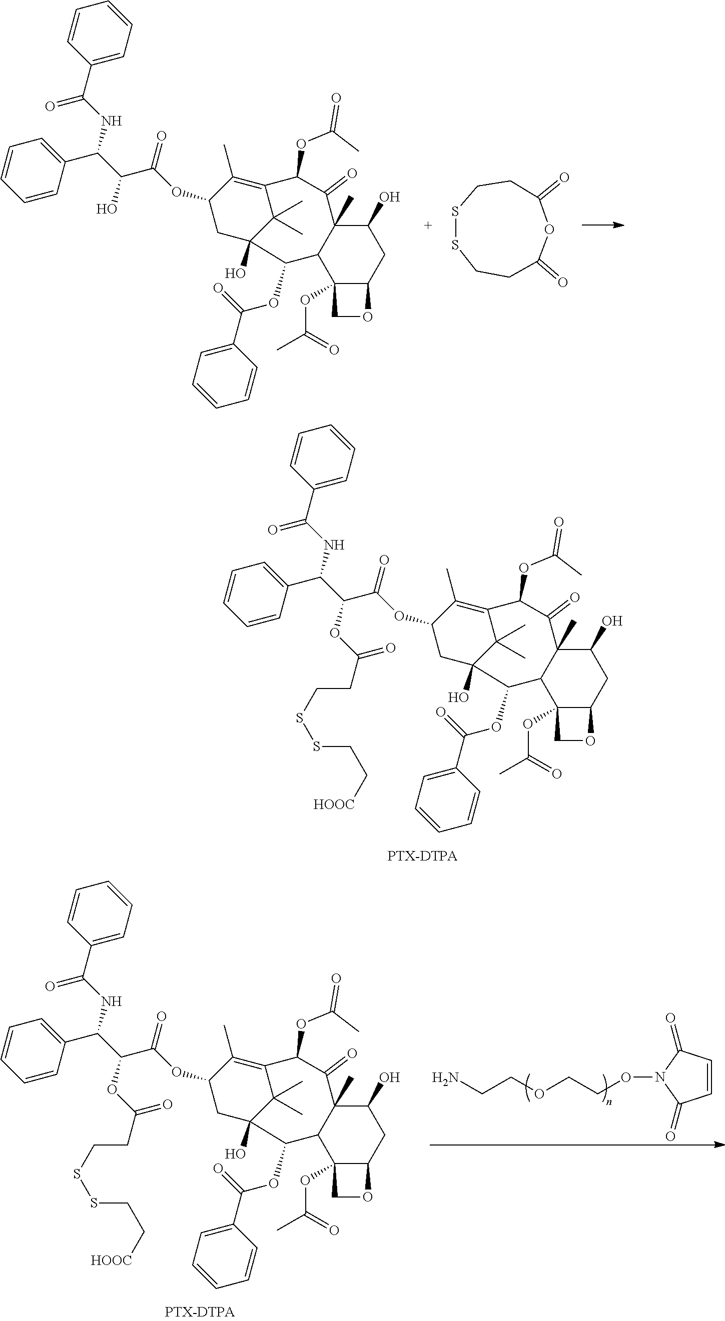

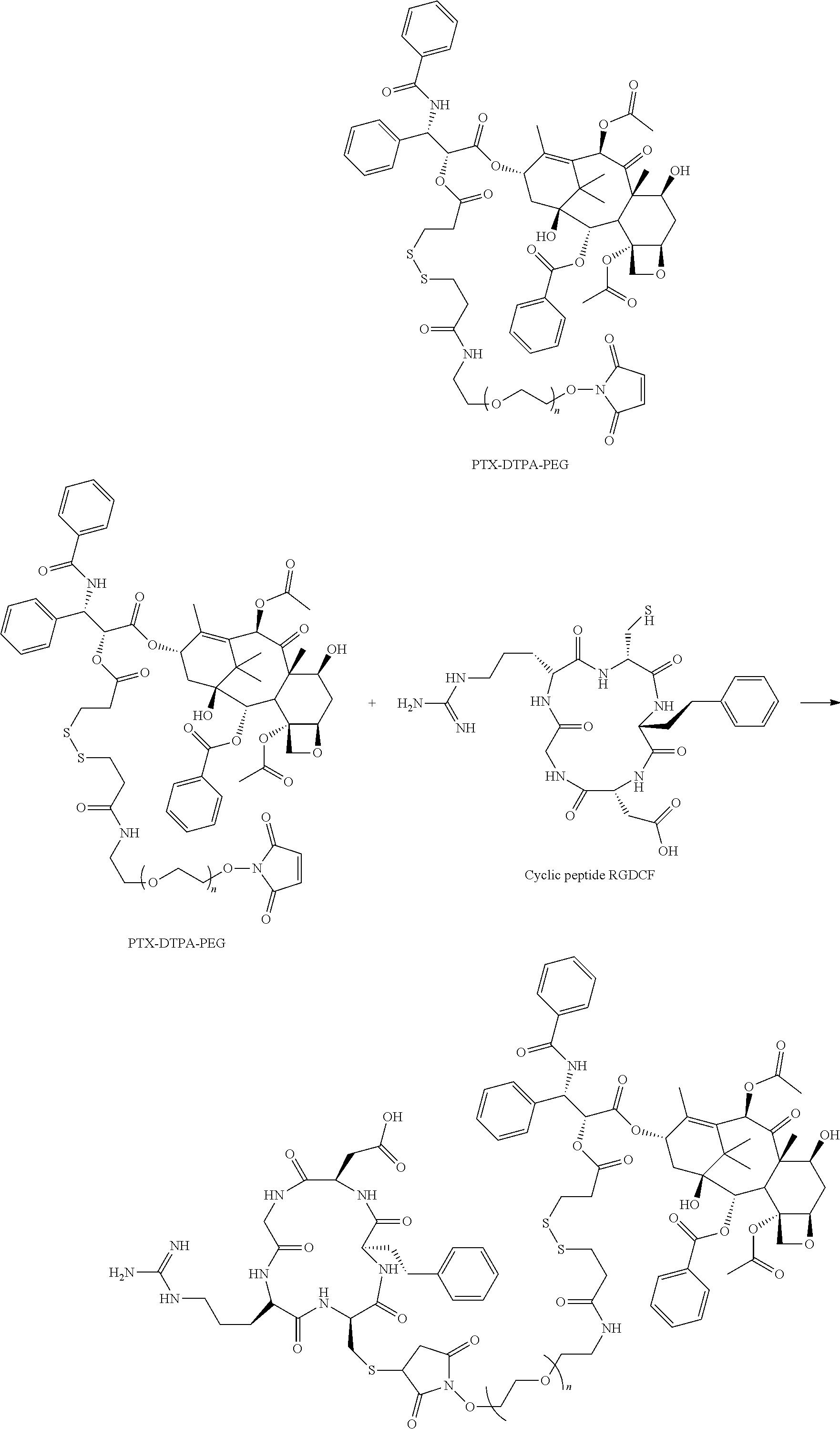

Further, in step (1), the activator is 4-dimethylaminopyridine, and the stabilizer is hydroxybenzotriazole; the organic solvent is tetrahydrofuran;
and/or instep (2), the activator is N-hydroxysuccinimide, the condensation agent is dicyclohexyl carbodiimide, and the stabilizer is hydroxybenzotriazole; the organic solvent is dichloromethane;
and/or instep (3), the aqueous solvent is a PBS solution.

Further, the reaction in step (1) is carried out at the temperature of 20-30° C. for 20-30 h;
and/or instep (2), the reaction is performed as follows: PTX-DTPA is mixed with the activator and the stabilizer, to which is then added the condensation agent at 0° C. The mixture was allowed to react for 5-7 h to activate the carboxyl group, followed by addition of Maleimide-PEG-Amine, and then the reaction mixture is allowed to react for 20-30 h;
and/or the reaction in step (3) is carried out at 20-30° C. for 20-30 h.

Further, in step (1), the molar ratio of paclitaxel, dithiodipropionic acid anhydride, the activator and the stabilizer is 2:1:0.06:0.06;
and/or, in step (2), the molar ratio of PTX-DTPA, Maleimide-PEG-Amine, the activator, the stabilizer, and the condensation agent is 1:0.75:3:3:3;
and/or, in step (3), the molar ratio of PTX-DTPA-PEG and the cyclic peptide RGDCF is 5:4.

The present invention also provides a drug-loaded micelle formed by self-assembly of the paclitaxel prodrug.

The present invention also provides the use of the paclitaxel prodrug or drug-loaded micelles in the preparation of medicaments for treating cancers.

The beneficial effects of the present invention: for the paclitaxel prodrug of the present invention, a disulfide bond, a hydrophilic PEG chain, and a RGD peptide sequence are successfully introduced into the paclitaxel moiety; compared with normal tissues, tumor tissues contain higher concentrations of reactive oxygen species (ROS) and glutathione (GSH), which are approximately 100-1000 times higher in tumor tissue than in blood. Due to the potential difference, the disulfide bond is specifically broken at the tumor site, thereby reducing the toxic and side effects on normal organs and tissues. Meanwhile, PEG is a commercially available polymer approved by FDA and widely used in the field of biology, which can reduce the uptake of reticuloendothelial cells, thus improving the half-life of ionomers and the therapeutic index of medicaments or the medicaments combined with it. While, RGD is a common cell-targeting peptide. RGD peptides can specifically recognize integrin αvβ3, which is overexpressed in tumor endothelial cells and epithelial cells, and thus RGD peptides can effectively achieve targeted effects. Therefore, the paclitaxel prodrug of the present invention can effectively improve the solubility of paclitaxel, enhance its targeting, and reduce toxic and side effects;

Moreover, the paclitaxel prodrug of the present invention can self-assemble into micelles in an aqueous environment, with good in vitro stability. The introduced disulfide bond is conducive to the specific bond-breaking of the prodrug at the tumor sites, that can cause the disintegration of the prodrug micelles, damage the micelle morphology, and thereby exhibit good sustained-release effects, indicating a promising application prospects.

The “cyclic peptide RGDCF” of the present invention refers to a 5-membered cyclic peptide consisting of arginine, glycine, aspartic acid, phenylalanine and cysteine by the linkage of head-to-tail amide bonds.

“The cyclic peptide moiety with a sequence of RGDCF” refers to the moiety formed by removing one H from the aforementioned cyclic peptide RGDCF.

Obviously, based on the above content of the present invention, according to the common technical knowledge and the conventional means in the field, without department from the above basic technical spirits, other various modifications, alternations, or changes can further be made.

With reference to the following specific examples of the embodiments, the above content of the present invention is further illustrated. But it should not be construed that the scope of the above subject matter of the present invention is limited to the following examples. The techniques realized based on the above content of the present invention are all within the scope of the present invention.

### DESCRIPTION OF THE FIGURES

FIG. 1. The NMR spectra of the paclitaxel prodrug.

FIG. 2. The IR spectra of the paclitaxel prodrug.

FIG. 3. TEM images of paclitaxel prodrug micelles in aqueous solution. (A) PDP; (B) PDPR; (C) PAP; (D) PAPR.

FIG. 4. Changes in particle size of four prodrug micelles placed in vitro for 7 days. (A) PDP; (B) PDPR; (C) PAP; (D) PAPR.

FIG. 5. The time-dependent changes in particle size and PDI of the four prodrug micelles after the addition of glutathione.

FIG. 6. The release curves of paclitaxel prodrug. (A) PDP; (B) PDPR; (C) PAP; (D) PAPR.

### EXAMPLES

The raw materials and equipment used in the present invention are known products obtained by purchasing commercially available products

### Example 1. Synthesis of the Paclitaxel Prodrug paclitaxel-dithiodipropionic acid-polyethylene glycol-RGD Peptide (PTX-DTPA-PEG-RGD, PDPR) According to the Present Invention

### 1. Synthesis of paclitaxel-dithiodipropionic acid (PTX-DTPA)

Paclitaxel (0.1 g, 0.12 mmol) and dithiodipropionic acid anhydride (0.12 g, 0.60 mmol) were dissolved in dry tetrahydrofuran, to which were added 4-dimethylaminopyridine (0.04 g, 0.36 mmol) and 1-hydroxybenzotriazole (0.05 g, 0.36 mmol), and then the reaction was stirred at room temperature for 24 h. After completion of the reaction, tetrahydrofuran was removed by evaporation with rotary evaporator, and then to the residue, was added water. The resultant solution was extracted three times with ethyl acetate, and finally purified over column chromatography (trichloromethane:methanol=50:1).

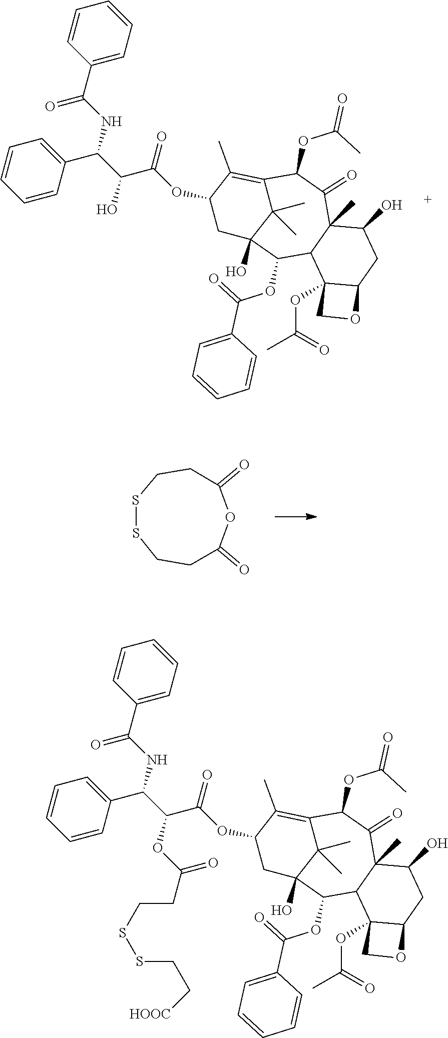 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O ; O=C1CCSSCCC(=O)O1 ; CC(=O)O[C@H]1C(=O)[C@@]2(C)C([C@H](OC(=O)c3ccccc3)[C@]3(O)C[C@H](OC(=O)[C@H](OC(=O)CCSSCCC(=O)O)[C@@H](NC(=O)c4ccccc4)c4ccccc4)C(C)=C1C3(C)C)[C@]1(OC(C)=O)CO[C@@H]1C[C@@H]2O]

### 2. Synthesis of paclitaxel-dithiodipropionic acid-polyethylene glycol (PTX-DTPA-PEG,PDP)

Paclitaxel-dithiodipropionic acid (PTX-DTPA) (0.039 g, 0.04 mmol), N-hydroxysuccinimide (0.014 g, 0.12 mmol), and 1-hydroxybenzotriazole (0.016 g, 0.12 mmol) were dissolved in dichloromethane, to which was added the solution of dicyclohexylcarbodiimide (0.024 g, 0.12 mmol) in dichloromethane dropwise in an ice salt bath, and then the reaction mixture was allowed to react for 6 h, to activate the carboxyl group. Then, Maleimide-PEG-Amine (0.06 g, 0.03 mmol) with a molecular weight of 2000 was added, and the reaction was allowed to react for 24 h, followed by filtering. After that, the resultant solution was washed three times with water, and purified over column chromatography (chloroform:methanol=30:1).

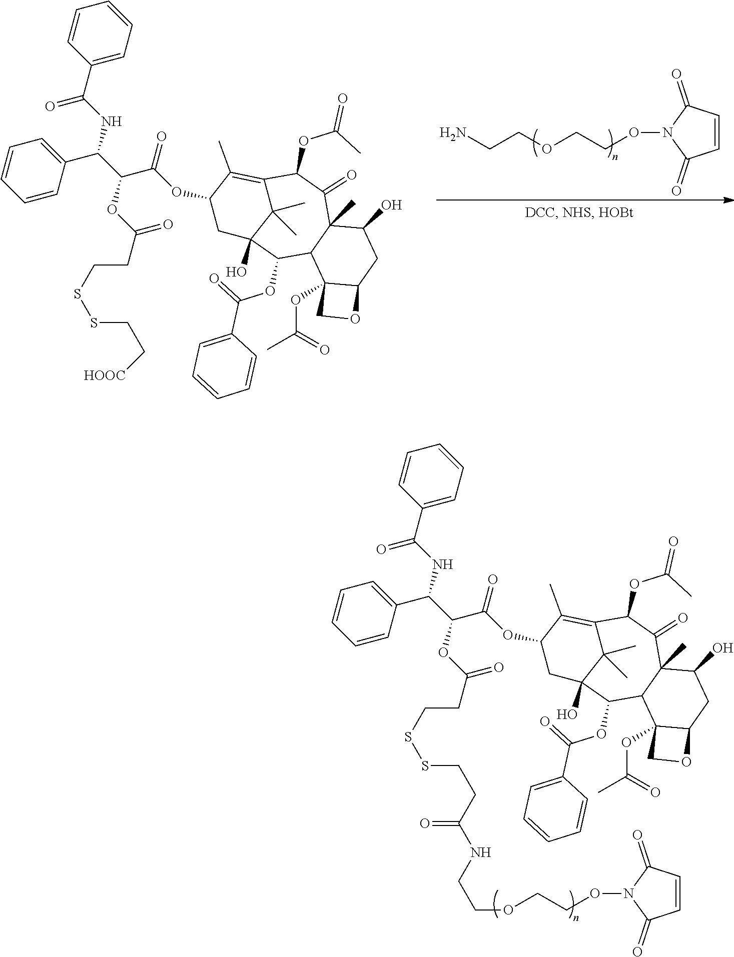

### 3. Synthesis of paclitaxel-dithiodipropionic acid-polyethylene glycol-RGD Peptide (PTX-DTPA-PEG-RGD, PDPR)

The thiol group in RGD was coupled with the maleimide in PTX-DTPA-PEG-Mal by thiol-maleimide Michael addition reaction, to synthesize PTX-DTPA-PEG-RGD. The cyclic peptide RGD (0.002 g, 0.004 mmol) and PTX-DTPA-PEG (0.015 g, 0.005 mmol) were dissolved in PBS (pH7.4), and then the reaction was allowed to react at room temperature for 24 h, to prepare the prodrug PDPR of the present invention.

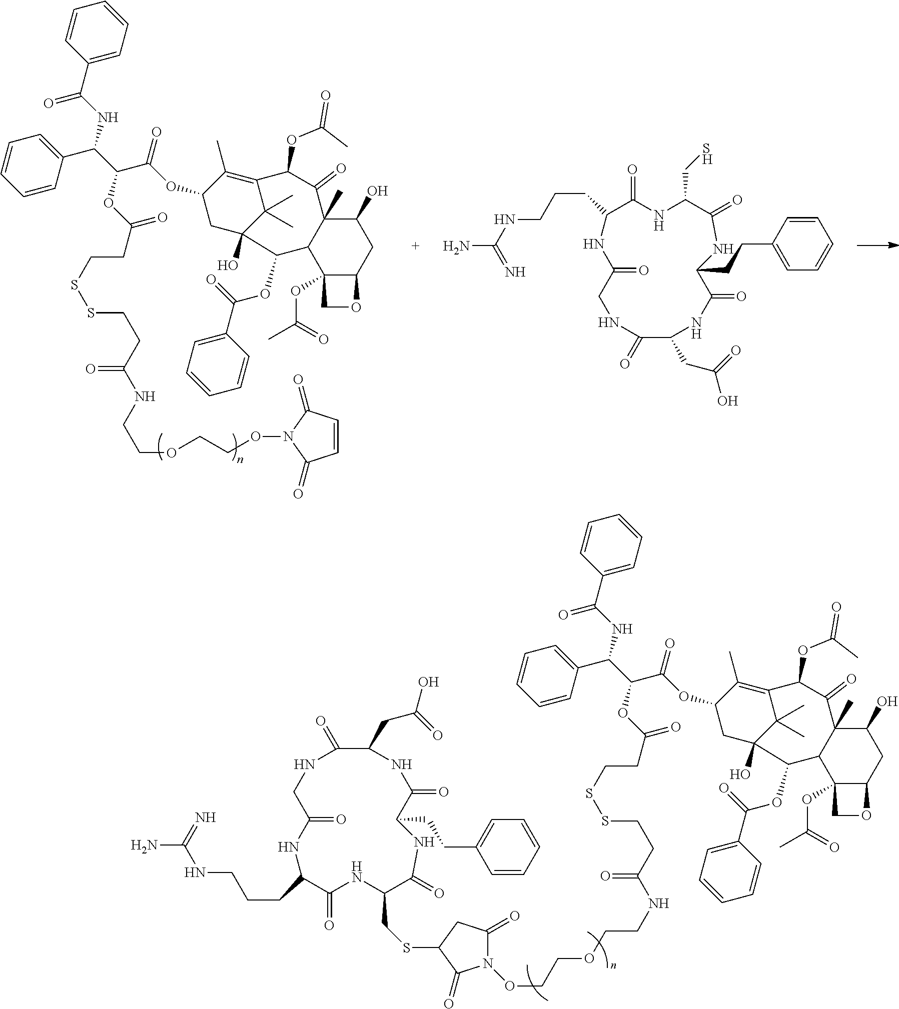

### Example 2. Preparation of Paclitaxel Prodrug Micelles According to the Present Invention

Preparation of micelles using the method of solvent evaporation: Firstly, 10 mg of the prodrug prepared in Example 1 was fully dissolved in 1 mL of tetrahydrofuran. The solution was drop added to the deionized water at a rate of one drop/30 s under stirring, and then the resultant solution was stirred overnight to evaporate the solvent and obtain the prodrug micelles. The micelle solution was centrifuged at a speed of 3000 r/min for 20 min, and then filtered with a 0.45 μm filter, to remove the precipitation. The prepared micelles were stored at 4° C.

### Comparative Example 1. Preparation of paclitaxel-dithiodipropionic acid-polyethylene glycol-RGD Peptide (PTX-DTPA-PEG-RGD, PDPR) and Micelles Thereof

PDP was prepared by referring to steps 1 and 2 of Example 1, while PDP drug-loaded micelles were prepared by referring to the method of Example 2.

### Comparative Example 2. Preparation of paclitaxel-adipic acid-polyethylene glycol (PTX-A-PEG, PAP) and Micelles Thereof

### 1. Synthesis of paclitaxel-adipic acid (PTX-A)

Substituting dithiodipropionic acid anhydride with adipic anhydride, paclitaxel (0.1 g, 0.12 mmol) and adipic anhydride (0.03 g, 0.24 mmol) were dissolved in dichloromethane, to which were then added 2,2-dihydroxymethylpropionic acid (0.04 g, 0.36 mmol) and 1-hydroxybenzotriazole (0.05 g, 0.36 mmol), and then the reaction mixture was allowed to react at room temperature for 24 h under stirring. After completion of the reaction, the reaction solution was washed three times by adding deionized water, and purified over column chromatography (trichloromethane:methanol=50:1).

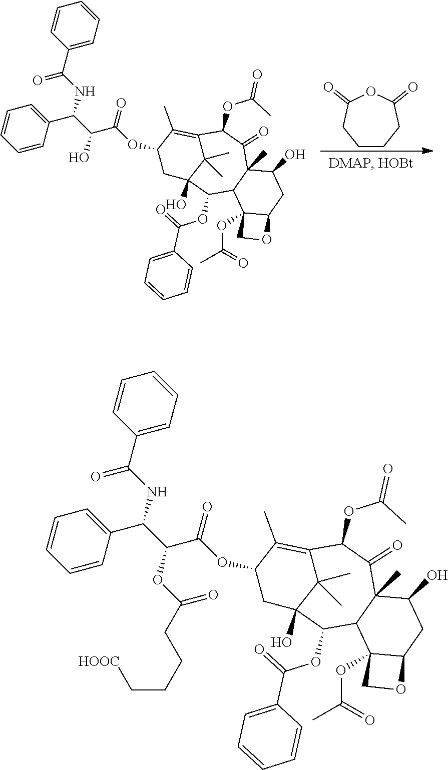 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O ; O=C1CCCCC(=O)O1 ; CC(=O)O[C@H]1C(=O)[C@@]2(C)C([C@H](OC(=O)c3ccccc3)[C@]3(O)C[C@H](OC(=O)[C@H](OC(=O)CCCCC(=O)O)[C@@H](NC(=O)c4ccccc4)c4ccccc4)C(C)=C1C3(C)C)[C@]1(OC(C)=O)CO[C@@H]1C[C@@H]2O]

### 2. Synthesis of paclitaxel-adipic acid-polyethylene glycol (PTX-A-PEG, PAP)

Paclitaxel-adipic acid (0.039 g, 0.04 mmol), N-hydroxysuccinimide (0.014 g, 0.12 mmol), and 1-hydroxybenzotriazole (0.016 g, 0.12 mmol) were dissolved in dichloromethane, to which was added the solution of dicyclohexylcarbodiimide (0.024 g, 0.12 mmol) in dichloromethane dropwise in an ice salt bath, and then the reaction mixture was allowed to react for 6 h, to activate the carboxyl group. Then, Maleimide-PEG-Amine with a molecular weight of 2000 (0.06 g, 0.03 mmol) was added. After 24 h of reaction, the reaction solution was filtered and then washed three times with deionized water, followed by purification over column chromatography (trichloromethane:methanol=30:1).

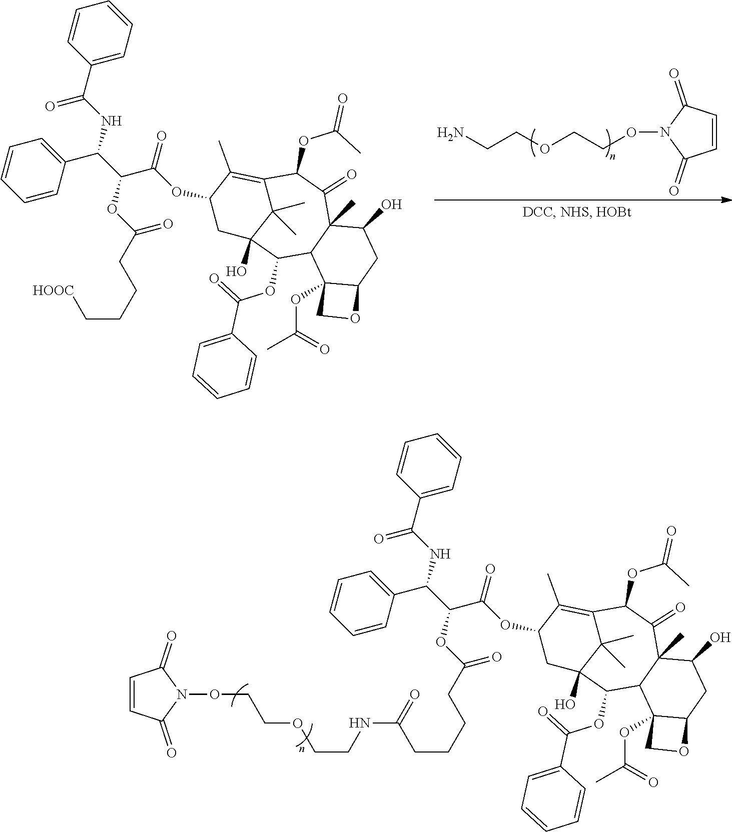

With reference to the method of Example 2, PAP drug-loaded micelles were prepared.

### Comparative Example 3. Preparation of paclitaxel-adipic acid-polyethylene glycol-RGD peptide (PTX-A-PEG-RGD, PAPR) and Micelles Thereof

RGD (0.002 g, 0.004 mmol) and PAP (0.010 g, 0.005 mmol) prepared in Comparative Example 2 were dissolved in PBS (pH 7.4) and then reacted at room temperature for 24 h.

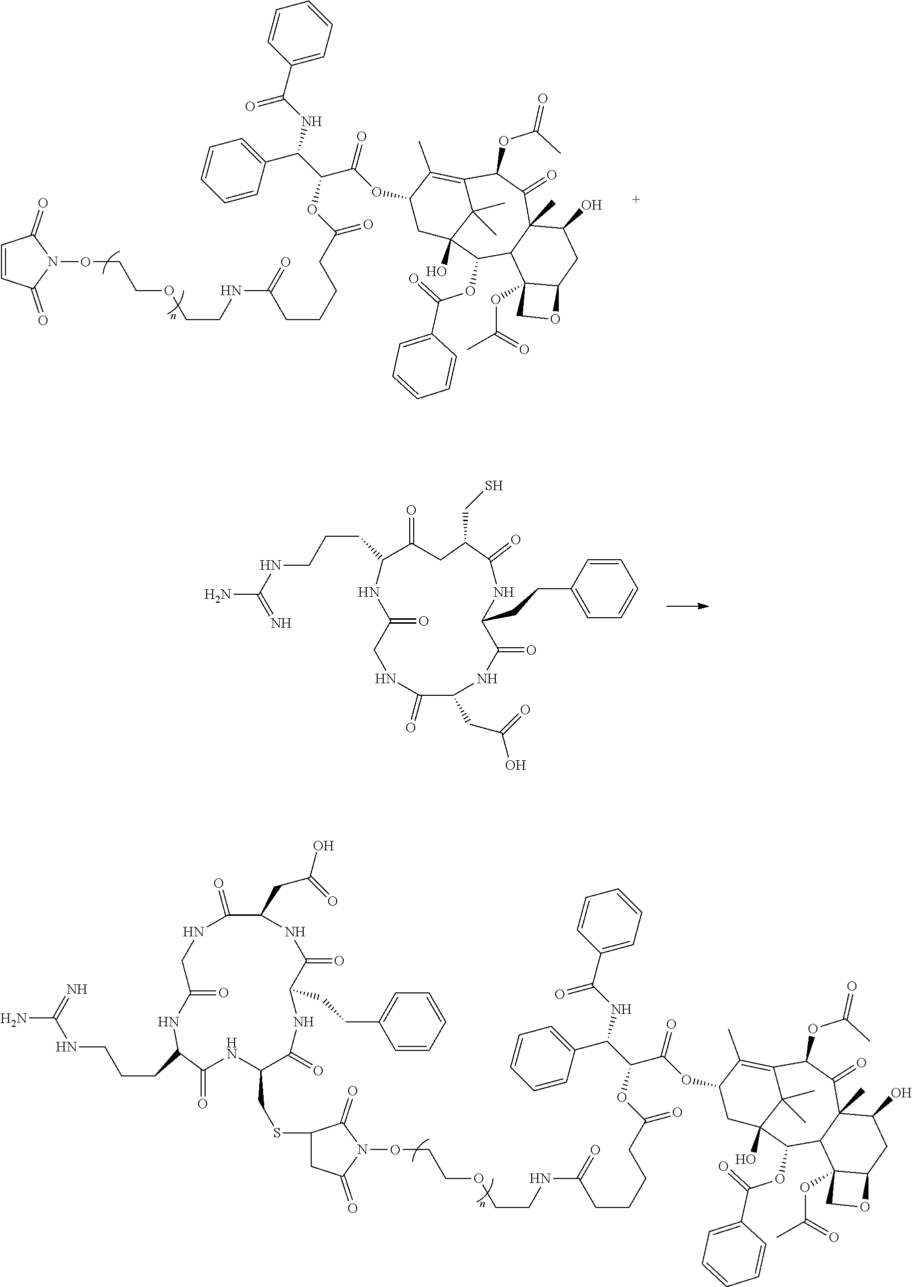

With reference to the method of Example 2, PAPR drug-loaded micelles were prepared.

The beneficial effects of the present invention were demonstrated with reference to Experimental Examples.

The characterization methods involved were as follows:

### 1. Characterization of the Structures of Compounds

### 1.1 Mass Spectrum (MS)

MS was measured on LCMS-2020 low resolution HPLC-MS using atmospheric pressure chemical ionization (positive ion mode).

### 1.2 Nuclear Magnetic Resonance Spectroscopy (NMR)

The test substance was dissolved in deuterated DMSO (DMSO-d₆, internal standard: tetramethylsilane (TMS)), and measured with a nuclear magnetic resonance instrument (400 MHZ, Bryker), to obtain the proton nuclear magnetic resonance (¹H NMR).

### 1.3 Fourier Transform Infrared Spectroscopy (FTIR)

The dried polymer was blended with completely dried KBr powder under infrared light and pressed into tablets. Infrared spectral data were collected using the Thermo Scientific Nicolet-iS50 (Thermo Electron Corporation, USA) infrared spectrometer, with a collection range of 4000-5000 cm⁻¹, 32 tests, and a resolution of 4 cm⁻¹.

### 2. Characterization of Micelles

### 2.1 Particle Size and Zeta Potential

The particle size, particle size distribution and zeta potential of micelles were measured by Zetasizer Nano ZS Dynamic light scattering (DLS) potentiometer (Malvern, UK) at 25° C. and 90° scattering angle.

### 2.2 Transmission Electronmicroscopy (TEM)

A small amount of micellar solution fully mixed under ultrasonic was added dropwise onto the copper mesh coated with Formvar film, and then dyed with 1% (w/v) phosphotungstic acid staining solution for 3 min. Subsequently, the excess liquid was absorbed with filter paper, and the copper mesh was allowed to dry naturally. Then, Tecnai G2 F20 S-TWIN transmission electron microscopy (FEI Corporation, USA) was used to observe the micellar morphology, and the acceleration voltage was 75 KV.

### 2.3 Study on the Stability of Micelles

In order to study the stability of micelles in vitro, the micellar solution was dropped into PBS simulating the human physiological environment, or a certain amount of glutathione was added. All samples were placed in a 37° C. constant temperature shaker (110 rpm), and the stability of micelles was studied by dynamic light scattering at the pre-determined time points.

2.4 Release performance of prodrug

The testing instrument was Agilent 1260 series (USA), with a reversed phase C18 column (4.6×100 mm). The mobile phase was acetonitrile/water (80/20), the column temperature was 30° C., the flow rate was 1.0 mL/min, and the detection wavelength was 227 nm.

Firstly, HPLC was used to detect the response of PTX in this mobile phase. After accurately weighing a certain amount of paclitaxel, PTX was prepared with the mixed solvent of acetonitrile and ultrapure water (9/1, v/v) into a series of standard solutions at different concentrations. Linear regression was performed between the measured response peak area A and concentration C, to obtain the standard curve of the PTX solution. The curve equation was fitted, and the concentration-peak area curve equation of PTX was Y=25.025X, with a correlation coefficient of R²=0.9996.

3 mL of drug-loaded micelles were loaded into a dialysis bag (MWCO=1000), and the release medium was PBS containing 30% ethanol (V/V) (pH 5.0 or 7.4). The release of paclitaxel from self-assembled nanoparticles of prodrug was investigated with or without the addition of glutathione (10 mM GSH). The specific procedures are as follows: 3 mL of prodrug nanoparticle solution (1 mg/mL) was placed into a dialysis bag (MW1000) and added to 30 mL of the release media, and then the entire system was transferred into a constant temperature shaker (37° C., 110 rpm). 1 mL of release solution was collected at different pre-determined time points, taking 3 parallel samples at each time point. After sampling, the same volume of pure PBS or PBS containing 10 mM GSH (10 Mm) was supplemented, and the drug concentration was determined by HPLC. The cumulative release was calculated using the following formula:

$$R = {V}_{i} {C}_{i} + \sum ( {V}_{sj} {C}_{j} )$$

wherein R is the cumulative release amount; Vi is the total volume of the solution before the i-th sampling; Ci is the total concentration of the solution before the i-th sampling; Vsj is the jth sampling volume; Cj is the total concentration of the solution before the jth sampling, i=2, 3, 4 . . . ; j=i−1.

### Experimental Example 1: Characterization Results of the Structure, the Micelle Structure, and the Stability of the Paclitaxel Prodrug According to the Present Invention

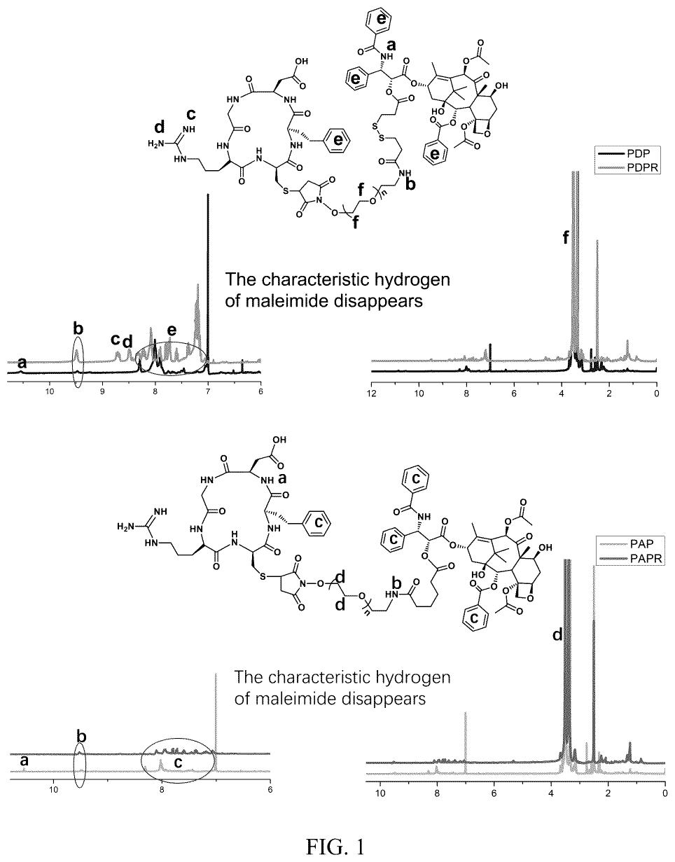

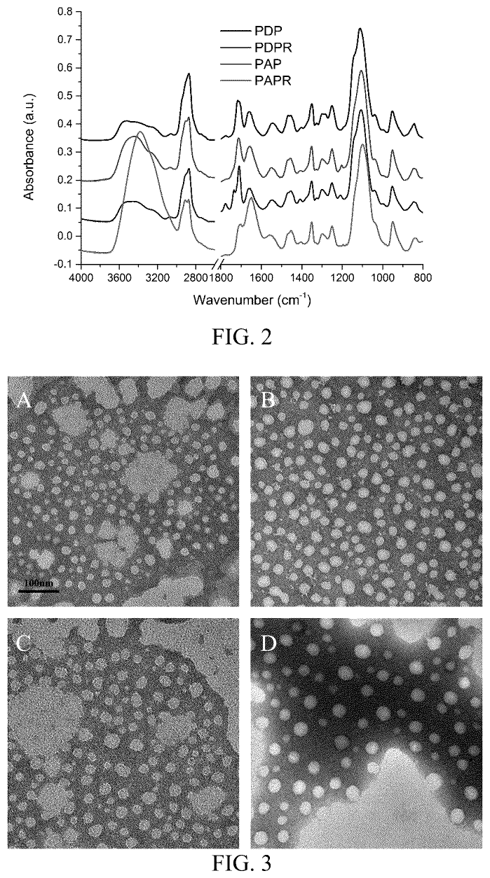

According to the NMR spectra in FIG. 1 and the IR spectra in FIG. 2, together with the molecular weight results of the MS for each prodrug, it could be confirmed that the paclitaxel prodrug of the present invention and each prodrug of Comparative Examples had been successfully synthesized.

The characterization results of micelles are shown in Table 1. The particle sizes of the four prodrug micelles were not significantly different, all greater than 100 nm; Zeta potential could be used to evaluate the charge and stability of micelle systems, and if the absolute value of Zeta potential was high and the repulsion between colloidal particles was large, the dispersion state tended to be stable. The absolute values of Zeta potential for PDP of Comparative Example 1 and PAP of Comparative Example 3 were relatively larger, which might be due to the reduction of electronegativity caused by the guanidino group of RGD peptide linked in Example 1 and Comparative Example 4. However, collectively, based on the Zeta potential measurement results of micelles, the Zeta potential of all micelles was ranged from-5 mV to-20 mV, corresponding to higher absolute values, which was beneficial for the stability of micelles.

TABLE 1
Micelle properties of PDP, PDPR, PAP,
and PAPR in aqueous solutions.

| Samples | Size(nm) | PDI | Zeta Potential(mV) |
|---|---|---|---|
| PDP | 129.07 ± 1.76 | 0.166 ± 0.003 | −18.1 ± 0.8 |
| PDPR | 114.63 ± 1.53 | 0.065 ± 0.012 | −7.38 ± 0.8 |
| PAP | 109.43 ± 0.94 | 0.103 ± 0.037 | −13.2 ± 0.6 |
| PAPR | 117.93 ± 5.57 | 0.110 ± 0.002 | −9.91 ± 0.3 |

TEM was used to observe the morphology of micelles, and as shown in FIG. 3, the morphology of each prodrug micelle was relatively regular and spherical, with a uniform distribution. The size based on DLS decreased due to the influence of water environment, which was about 30 nm. Overall, the PDPR micelles of Example 1 according to the present invention had uniform particle size and dispersion.

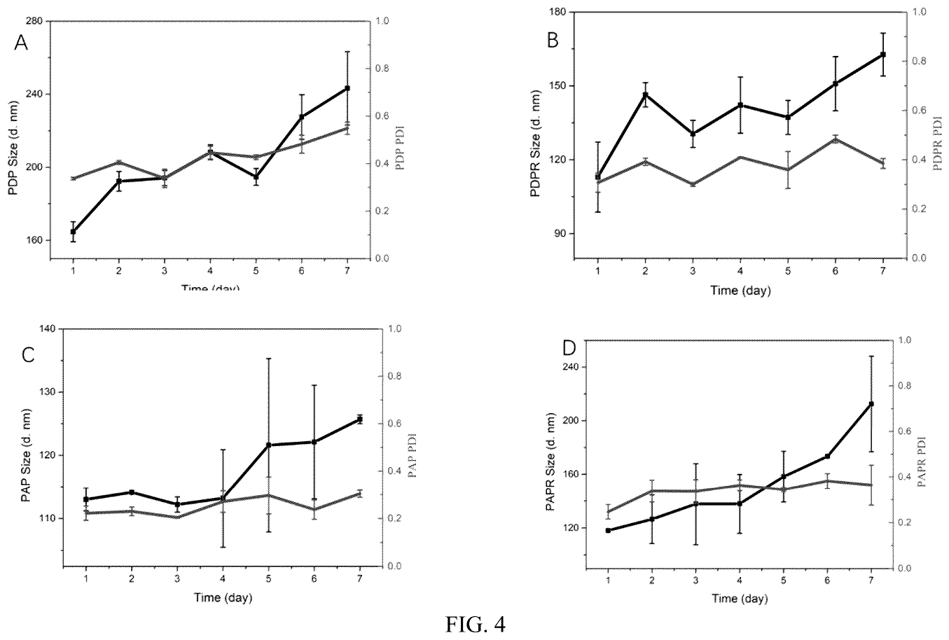

After being placed in a shaker for 7 days, the particle size and PDI of each micelle slightly increased, but all four prodrug micelles showed certain stability (FIG. 4).

### Experimental Example 2: Characterization Results of the Micelle Response and Sustained-Release Performance of the Paclitaxel Prodrug According to the Present Invention

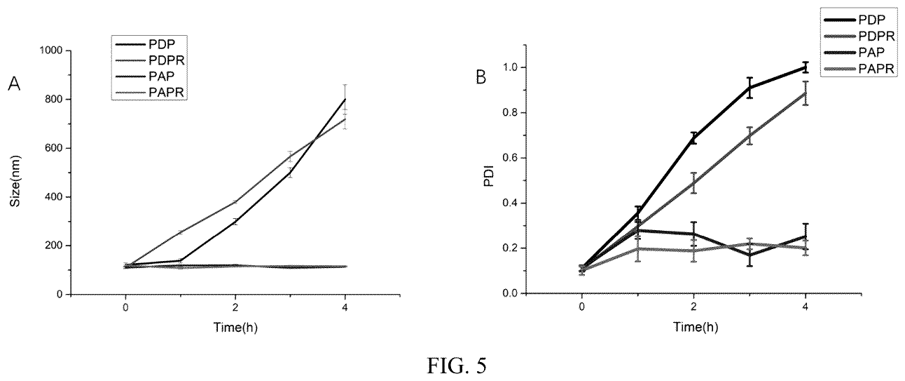

Glutathione (GSH) was added to simulate the tumor microenvironment, so as to promote the disulfide bond breaking. As shown in FIG. 5, the prodrug micelles with disulfide bond PDP (Comparative Example 1) and PDPR (Example 1) significantly disintegrated due to the breaking of disulfide bond, leading to changes in particle size and PDI; the prodrug micelles without disulfide bonds could still maintain a stable state, with almost no change in particle size. As shown in FIG. 2, after being placed in a shaker for 7 days, the particle size and PDI slightly increased, but the four types of prodrug micelles showed certain stability.

Further, the release of original paclitaxel from prodrugs linked by chemical bonds was necessary for paclitaxel prodrug to reach the tumor sites and play roles. However, the release rate had a significant impact on the therapeutic effect of tumors. If the drug release rate was too fast, the majority of drugs had already been released to normal tissues before reaching the tumor tissues, that would have significant toxic and side effects on normal tissues, and cause significant loss of drug metabolism in the body and poor therapeutic effects; if the release rate was too slow, the therapeutic activity of the medicament might be reduced, not achieving good therapeutic effects. Therefore, the release of chemically bonded prodrugs in vitro was an important evaluation criterion for the anti-tumor effects of medicaments.

The theoretical drug loading rate of the prodrug according to the present invention could be calculated by the molecular weight:

$$Drug loading rate = \frac{The molecular weight of PTX}{The molecular weight of paclitaxel prodrug} \times 100 \%$$

After calculation, the theoretical drug loading rate of PDP is 28.2%, PDPR is 23.7%, PAP is 28.8%, and PAPR is 24.1%.

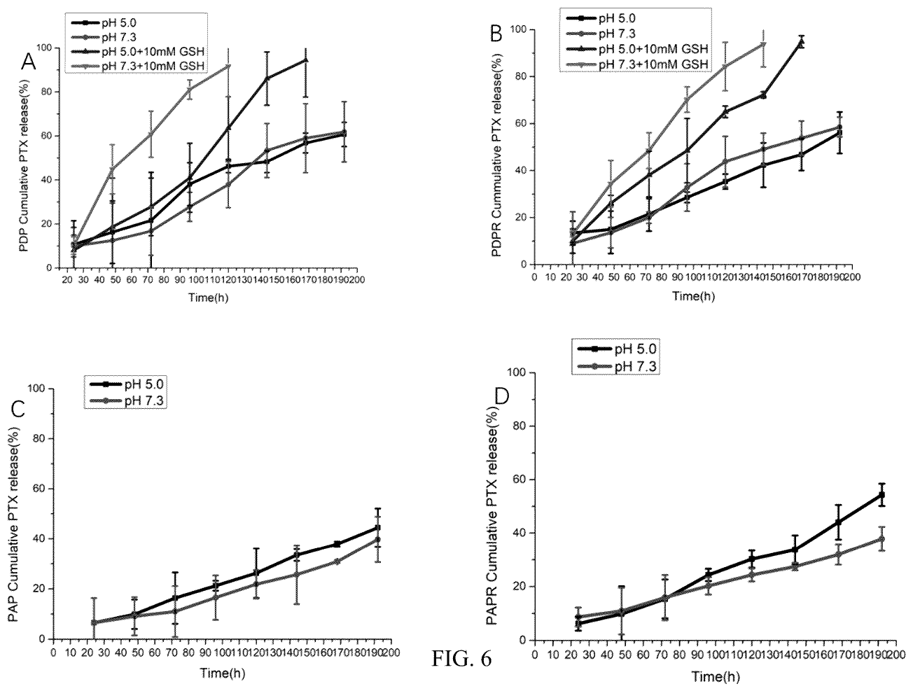

Based on the above, the hydrolysis release curves of the prodrugs were measured and calculated, as shown in FIG. 6. It could be found that under neutral conditions of pH 7.4 or acidic conditions of pH 5.0, without the addition of GSH, the release rates of the four prodrugs were slower, and the difference in release rates between neutral and acidic environments was also very small. After 200 hours, only about 50% of paclitaxel was hydrolyzed from the polymer in the micelles of the four prodrugs. However, after the addition of GSH, the release rate of PDP and PDPR was significantly accelerated due to the disulfide bond breaking. Under the conditions of neutral pH 7.4 and adding GSH (almost complete release after 120 hours), the release rate was slightly faster than that under the conditions of acidic pH 5.0 and adding GSH (almost complete release after 144 hours), confirming that the introduction of disulfide bonds in the prodrug of the present invention promoted the drug release at the tumor sites.

In summary, the present invention provided a paclitaxel prodrug, and a disulfide bond, a hydrophilic PEG chain, and a RGD peptide sequence were successfully introduced into the paclitaxel moiety, thereby, the prodrug could effectively improve the solubility of paclitaxel, enhance its targeting, and reduce toxic and side effects. Moreover, the paclitaxel prodrug of the present invention could self-assemble into micelles in an aqueous environment, with good in vitro stability. The introduced disulfide bond was conducive to the specific bond-breaking of the prodrug at the tumor sites, that could cause the disintegration of the prodrug micelles, damage the micelle morphology, and thus exhibit good sustained-release effects, having a promising application prospects.

1. A paclitaxel prodrug, characterized in that its structure comprises a disulfide bond, a polyethylene glycol molecular chain, and an RGD peptide chain.

2. A paclitaxel prodrug according to claim 1, characterized in that the structure includes a cyclic peptide moiety with the sequence RGDCF.

3. A paclitaxel prodrug according to claim 2, characterized in that the structure is: 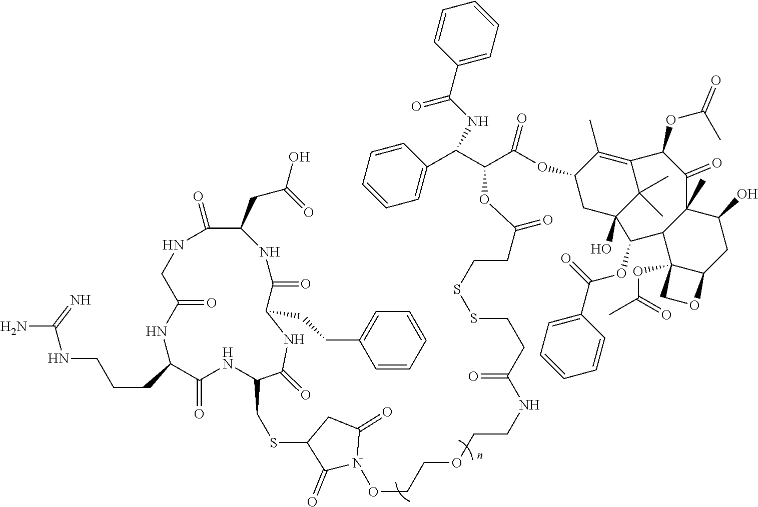
wherein n is any value between 40 and 50.

4. A paclitaxel prodrug according to claim 1, characterized in that the number-average molecular weight of the polyethylene glycol (PEG) molecular chains is 2000.

5. A preparation method for the paclitaxel prodrug according to claim 1, characterized in that the method comprises the following steps:
(1) Paclitaxel reacts with dithiodipropionic acid anhydride in an organic solvent in the presence of an activator and a stabilizer, to prepare PTX-DTPA;
(2) PTX-DTPA reacts with Maleimide-PEG-Amine in an organic solvent in the presence of an activator, a stabilizer and a condensation agent, to prepare PTX-DTPA-PEG;
(3) PTX-DTPA-PEG and cyclic peptide RGDCF react in aqueous solvents, to prepare the paclitaxel prodrug;
The reaction formula is as follows:

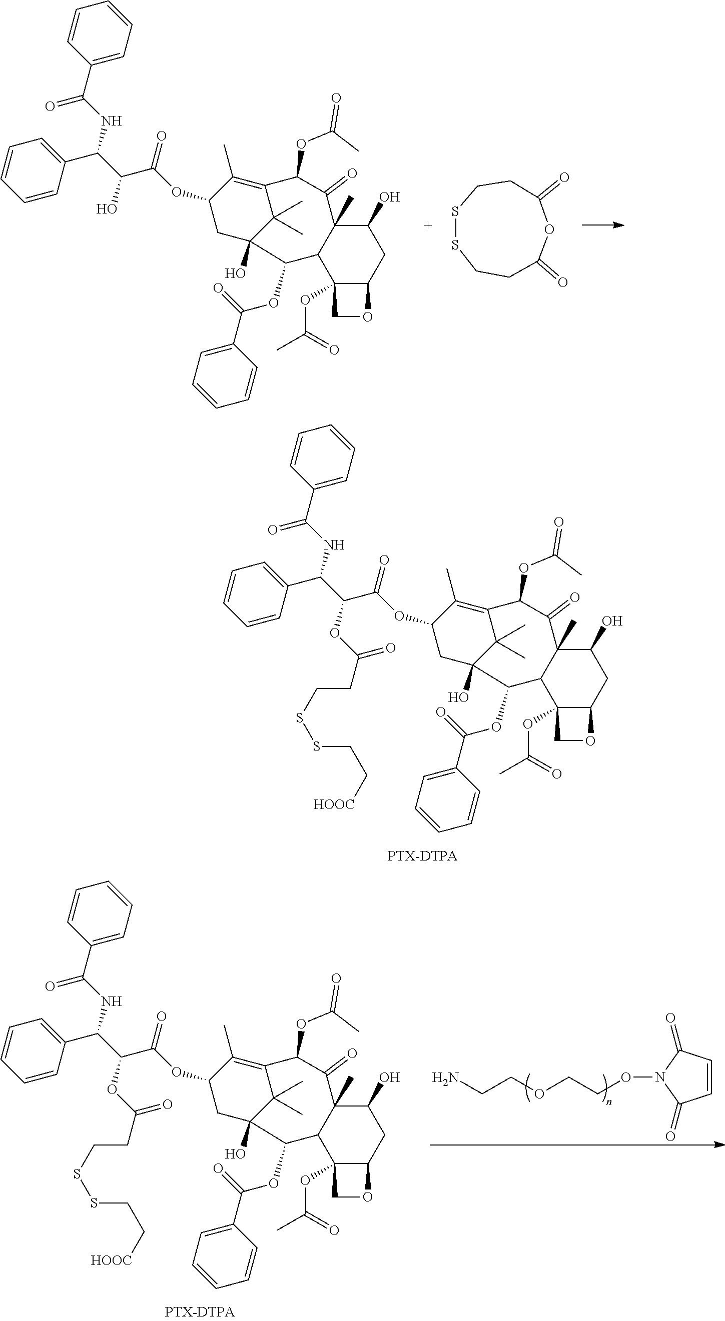

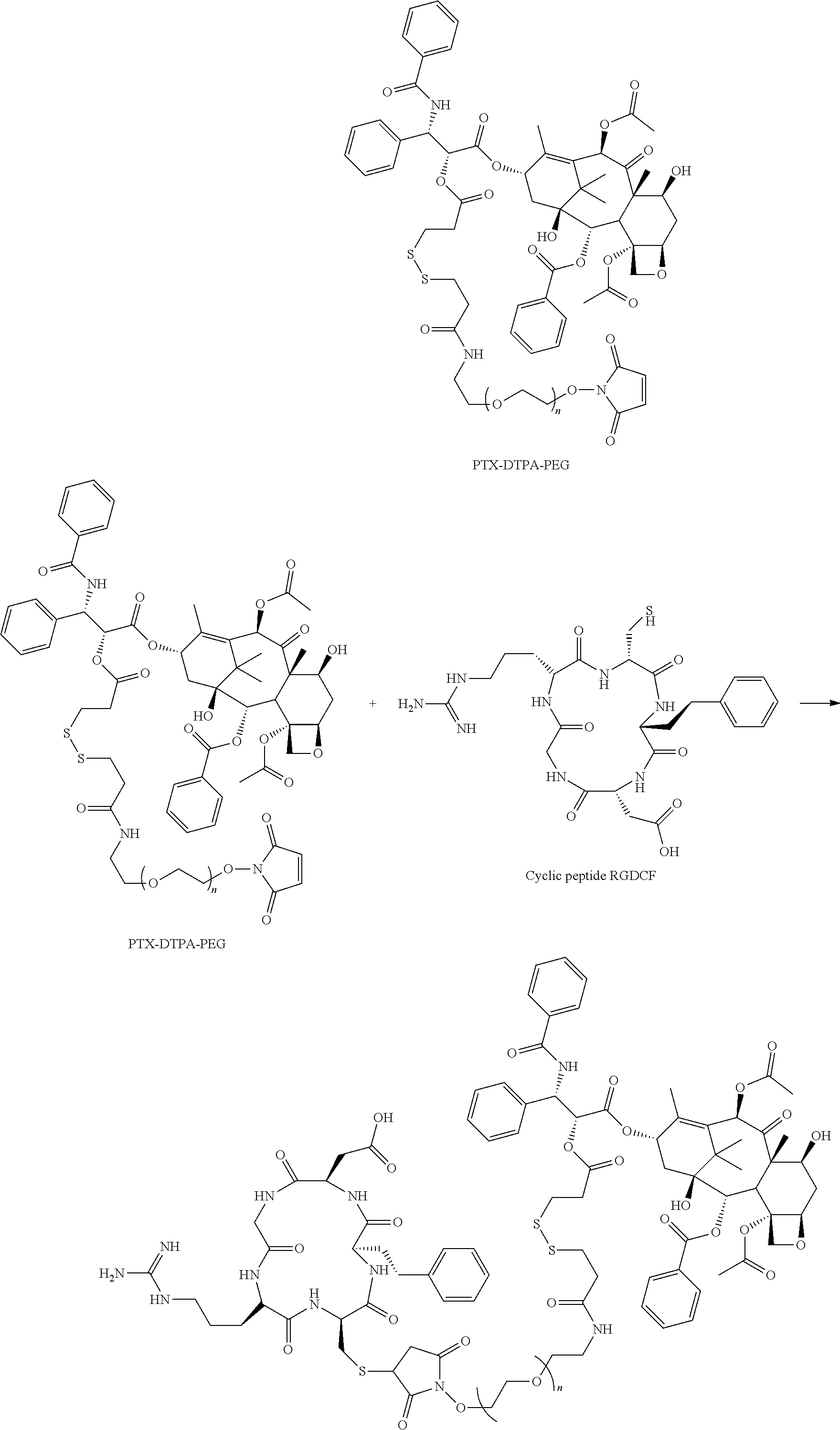

6. The preparation method according to claim 5, characterized in that in step (1), the activator is 4-dimethylaminopyridine, and the stabilizer is hydroxybenzotriazole; the organic solvent is tetrahydrofuran;
and/or instep (2), the activator is N-hydroxysuccinimide, the condensation agent is dicyclohexyl carbodiimide, and the stabilizer is hydroxybenzotriazole; the organic solvent is dichloromethane;
and/or instep (3), the aqueous solvent is a PBS.

7. The preparation method according to claim 5, characterized in that the reaction in step (1) is carried out at the temperature of 20-30° C. for 20-30 h;
and/or instep (2), the reaction is performed as follows: PTX-DTPA is mixed with the activator and the stabilizer, to which is then added the condensation agent at 0° C. The mixture was allowed to react for 5-7 h to activate the carboxyl group, followed by addition of Maleimide-PEG-Amine, and then the reaction mixture is allowed to react for 20-30 h;
and/or the reaction in step (3) is carried out at 20-30° C. for 20-30 h.

8. The preparation method according to claim 5, characterized in that in step (1), the molar ratio of paclitaxel, dithiodipropionic acid anhydride, the activator and the stabilizer is 2:1:0.06:0.06;
and/or, in step (2), the molar ratio of PTX-DTPA, Maleimide-PEG-Amine, the activator, the stabilizer, and the condensation agent is 1:0.75:3:3:3;
and/or, in step (3), the molar ratio of PTX-DTPA-PEG and the cyclic peptide RGDCF is 5:4.

9. A drug-loaded micelle, characterized in that it is formed by self-assembly of the paclitaxel prodrug according to claim 1.

10. The paclitaxel prodrug according to claim 1 for use in the preparation of medicaments for treating cancers.

11. The drug-loaded micelles according to claim 8 for use in the preparation of medicaments for treating cancers.
